(19) United States
(12) Patent Application Publication
(10) Pub. No.: US 2016/0089114 A1
(43) Pub. Date: Mar. 31, 2016

(54) ULTRASOUND SYSTEM, METHOD AND COMPUTER-READABLE STORAGE MEDIUM FOR PROVIDING DOPPLER IMAGE

(71) Applicant: Siemens Medical Solutions USA, Inc., Malvern, PA (US)

(72) Inventors: JoongHyuk Kim, Hoengseong-gun (KR); KwangJae Lee, Yongin-si (KR); SangHyuk Kim, Suwon-si (KR); JiHyun Kim, Seongnam-si (KR)

(21) Appl. No.: 14/821,565

(22) Filed: Aug. 7, 2015

(30) Foreign Application Priority Data
Sep. 30, 2014 (KR) 2014-0131424

(51) Int. Cl. A61B 8/08; A61B 8/06; A61B 8/14; A61B 8/00

(52) U.S. Cl. CPC A61B 8/5207; A61B 8/463; A61B 8/06; A61B 8/14; A61B 8/5223

(57) ABSTRACT

An ultrasound system, a method, and a computer-readable storage medium for providing a Doppler image of blood flow and a Doppler image of tissue movement are disclosed. The ultrasound system includes an ultrasound probe, a processor, and a display section. The ultrasound probe is configured to transmit ultrasound signals into a target object having blood flow and tissue, and receive ultrasound echo signals reflected from the target object. The processor is configured to acquire ultrasound data based on the ultrasound echo signals, generate a first Doppler image of the blood flow based on the ultrasound data, perform down-sampling on the ultrasound data to obtain down-sampled ultrasound data, and generate a second Doppler image indicating movement of the tissue based on the down-sampled ultrasound data. The display section is configured to display the first and second Doppler images.

### CROSS-REFERENCE TO RELATED APPLICATION

This application claims the benefit of Korean Patent Application No. 10-2014-0131424, filed on Sep. 30, 2014, in the Korean Intellectual Property Office, the disclosure of which is incorporated herein in its entirety by reference.

### TECHNICAL FIELD

The present disclosure relates to an ultrasound system, and more particularly, to an ultrasound system, method, and computer-readable storage medium for providing Doppler images (blood flow Doppler images and tissue Doppler images).

Ultrasound systems have been widely used in the medical field to obtain information within the human body due to their non-invasive and non-destructive nature. Since ultrasound systems make it possible to provide a high-resolution image of tissue of a living body in real time without a need for a surgical operation that dissects and observes the living body, their use has become very important in medical fields.

An ultrasound system transmits ultrasound signals into a living body and receives ultrasound signals (i.e., ultrasound echo signals) reflected from the living body, thereby generating ultrasound images of the living body. Ultrasound images include a B-mode (brightness mode) image showing reflection coefficients of ultrasound echo signals as a two-dimensional image, a Doppler image showing a velocity or direction of a moving target object within a living body as an image by using Doppler effect, an elastic image showing a difference in response characteristics of a tissue before and after applying stress to the target object as an image, and the like.

Meanwhile, the ultrasound system provides, as Doppler images, a blood flow Doppler image, which shows the velocity and/or direction corresponding to the movement of the blood flow as an image, and a tissue Doppler image, which shows the velocity corresponding to the movement of the tissue as an image. Particularly, the ultrasound system may display movement of the myocardium as a tissue Doppler image in a heart diagnosis application for medical diagnosis.

Generally, responses of the myocardium and the blood flow to ultrasound signals are different from each other in that the movement of the blood flow is faster than that of the myocardium but the ability of the blood flow to reflect ultrasound signals is weak. That is, the blood flow has a relatively higher velocity and lower signal strength as compared with the tissue, whereas the tissue has a relatively lower velocity and higher signal strength.

A range of velocities that can be shown in an ultrasound system is determined by a pulse repetition frequency. When a difference in velocity between the blood flow and the tissue becomes larger as in a heart diagnosis application, the difference between pulse repetition frequencies that should be used in a Doppler imaging mode for obtaining a blood flow Doppler image and a Doppler imaging mode for obtaining a tissue Doppler image also becomes larger. Accordingly, in order to simultaneously display the blood flow Doppler image and the tissue Doppler image, relevant data should be acquired by performing transmission and reception of ultrasound signals in accordance with pulse repetition frequencies corresponding to the respective Doppler imaging modes, and this leads to a problem where a loss in temporal resolution occurs.

### SUMMARY

The present disclosure provides an ultrasound system, method, and computer-readable storage medium for generating and providing a blood flow Doppler image and a tissue Doppler image by using ultrasound data for obtaining the blood flow Doppler image, without a loss in temporal resolution.

In one embodiment, an ultrasound system includes: an ultrasound probe configured to transmit ultrasound signals into a target object having blood flow and tissue, and receive ultrasound echo signals reflected from the target object; a processor configured to acquire ultrasound data based on the ultrasound echo signals, generate a first Doppler image of the blood flow based on the ultrasound data, perform down-sampling on the ultrasound data to obtain down-sampled ultrasound data, and generate a second Doppler image indicating movement of the tissue based on the down-sampled ultrasound data; and a display section configured to display the first Doppler image and the second Doppler image.

In another embodiment, a method of providing a Doppler image of a target object in an ultrasound system includes: transmitting ultrasound signals into a target object having blood flow and tissue; receiving ultrasound echo signals reflected from the target object; acquiring ultrasound data based on the ultrasound echo signals; generating a first Doppler image of the blood flow based on the ultrasound data; performing down-sampling on the ultrasound data to obtain down-sampled ultrasound data; generating a second Doppler image indicating movement of the tissue based on the down-sampled ultrasound data; and displaying the first Doppler image and the second Doppler image.

In yet another embodiment, a non-transitory computer-readable storage medium includes instructions that, when executed by a processor, cause the processor to perform operations of: generating ultrasound data based on ultrasound echo signals reflected from a target object having blood flow and tissue; generating a first Doppler image of the blood flow based on the ultrasound data; performing down-sampling on the ultrasound data to obtain down-sampled ultrasound data; generating a second Doppler image indicating movement of the tissue based on the down-sampled ultrasound data; and displaying the first Doppler image and the second Doppler image.

### BRIEF DESCRIPTION OF THE DRAWINGS

FIG. 1 is a block diagram schematically showing a configuration of an ultrasound system according to an embodiment of the present disclosure.

FIG. 2 is a block diagram schematically showing a configuration of a processor according to an embodiment of the present disclosure.

FIG. 3 is an exemplary illustration of storing ultrasound data according to an embodiment of the present disclosure.

FIG. 4 is a block diagram schematically showing a configuration of a Doppler image generating section according to an embodiment of the present disclosure.

FIGS. 5A and 5B are explanatory illustrations showing a relationship between pulse repetition frequencies for obtaining a blood flow Doppler image and a tissue Doppler image according to an embodiment of the present disclosure.

FIG. 6 is an exemplary illustration of a blood flow Doppler image and a tissue Doppler image that are displayed in an overlapped manner according to an embodiment of the present disclosure.

FIG. 7 is an exemplary illustration showing a blood flow Doppler image and a tissue Doppler image that are displayed side-by-side in a non-overlapped manner according to an embodiment of the present disclosure.

FIG. 8 is a flowchart illustrating a process of simultaneously providing a blood flow Doppler image and a tissue Doppler image according to an embodiment of the present disclosure.

### DETAILED DESCRIPTION

Hereinafter, embodiments of the present disclosure will be described with reference to the accompanying drawings. The term “section” used in these embodiments means a software component or hardware component, such as a field-programmable gate array (FPGA) and an application specific integrated circuit (ASIC). However, the “section” is not limited to software and hardware. The “section” may be configured to be in an addressable storage medium or may be configured to run one or more processors. Accordingly, as an example, the “section” includes components, such as software components, object-oriented software components, class components, and task components, as well as processors, functions, attributes, procedures, subroutines, segments of program codes, drivers, firmware, micro-codes, circuits, data, databases, data structures, tables, arrays, and variables. Functions provided in components and “sections” may be combined into a smaller number of components and “sections” or further subdivided into additional components and “sections.”

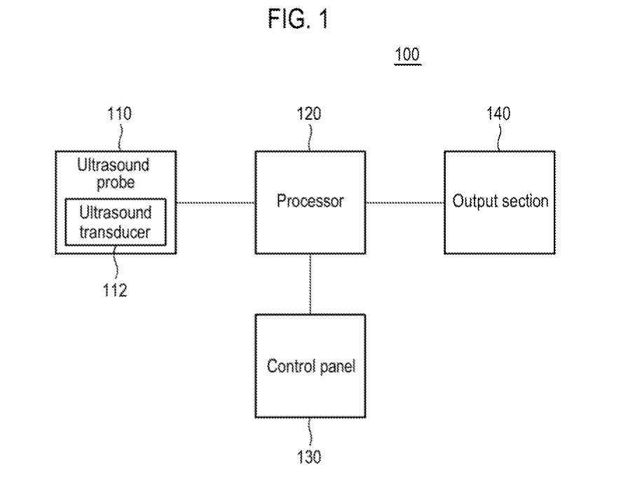

FIG. 1 is a block diagram schematically showing a configuration of an ultrasound system according to an embodiment of the present disclosure. Referring to FIG. 1, the ultrasound system 100 includes an ultrasound probe 110.

The ultrasound probe 110 transmits ultrasound signals into a living body (not shown) and receives ultrasound signals (i.e., ultrasound echo signals) reflected from the living body. The ultrasound probe 110 includes an ultrasound transducer 112 for reciprocally converting between the ultrasound signals and electrical signals, as shown in FIG. 1. The ultrasound transducer 112 converts the electrical signals into the ultrasound signals and transmits the converted ultrasound signals into the living body. In addition, the ultrasound transducer 112 receives ultrasound echo signals reflected from the living body and converts the received ultrasound echo signals into electrical signals (hereinafter, referred to as “reception signals”). The reception signals are analog signals. The ultrasound probe 110 maybe a convex probe, a linear probe, or any other.

The ultrasound system 100 further includes a processor 120. The processor 120 controls transmission of the ultrasound signals. In addition, the processor 120 generates electrical signals (hereinafter, referred to as “transmission signals”) for obtaining an ultrasound image and transmits the generated transmission signals to the ultrasound probe 110. The processor 120 also performs signal processing on the reception signals transmitted from the ultrasound probe 110 to generate an ultrasound image of the living body. The processor 120 will be described in detail below. The processor 120 includes a central processing unit (CPU), a graphic processing unit (GPU), a microprocessor, and the like.

The ultrasound system 100 further includes a control panel 130. The control panel 130 receives input information from a user and transmits the received input information to the processor 120. The control panel 130 is a component having various input devices installed therein for performing operations, such as selection of a diagnosis mode, control of a diagnostic operation, input of a command needed for diagnosis, signal control, output control and the like, and enables interfacing between the user and the ultrasound system. The control panel 130 is equipped with an input section such as a trackball, a keyboard, buttons, or the like. For example, the control panel 130 receives information from the user that sets a region of interest (ROI) for which a Doppler image is to be obtained onto a B-mode (brightness mode) image (hereinafter, referred to as “ROI setting information”), and transmits the received ROI setting information to the processor 120.

The ultrasound system 100 further includes an output section 140. The output section 140 outputs the ultrasound image generated from the processor 120. The output section 140 may also output the input information inputted through the control panel 130. The output section 140 may include a display section, a speaker, or the like.

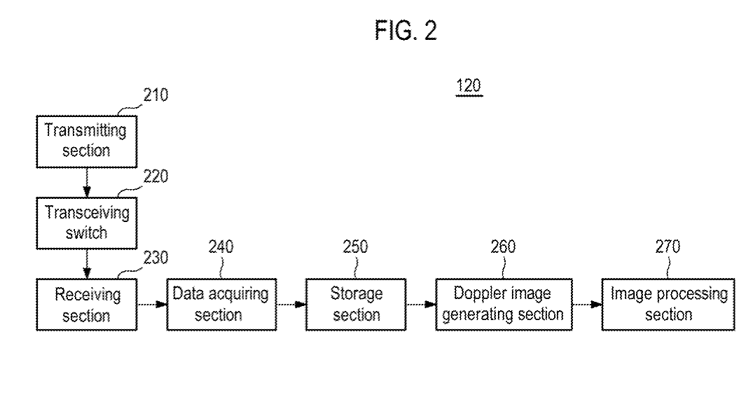

FIG. 2 is a block diagram schematically showing a configuration of the processor 120 according to an embodiment of the present disclosure. Referring to FIG. 2, the processor 120 includes a transmitting section 210. The transmitting section 210 generates transmission signals for obtaining an ultrasound image. In this embodiment, the transmitting section 210 generates transmission signals for obtaining a blood flow Doppler image based on an ensemble number and a pulse repetition frequency and transmits the transmission signals to the ultrasound probe 110 through a transceiving switch 220. In this embodiment, the pulse repetition frequency is a pulse repetition frequency adapted to obtain the blood flow Doppler image corresponding to a movement of the blood flow. The ensemble number represents the number of times ultrasound signals are transmitted and received for obtaining Doppler data corresponding to one scan-line. Accordingly, the ultrasound probe 110 converts the transmission signals provided from the transmitting section 210 into ultrasound signals and transmits the converted ultrasound signals into the living body, receives ultrasound echo signals reflected from the living body, and generates reception signals.

The processor 120 further includes the transceiving switch 220. The transceiving switch 220 functions as a duplexer to prevent the transmission signals of a high voltage output from the transmitting section 210 from affecting a receiving section 230 as described below. That is, when the ultrasound transducer 112 alternately performs transmission and reception, the transceiving switch 220 functions to appropriately switch the transmitting section 210 and the receiving section 230 to the ultrasound transducer 112.

The processor 120 further includes the receiving section 230. The receiving section 230 amplifies reception signals, which are radio frequency (RF) signals, provided from the ultrasound probe 110 through the transceiving switch 220 and then converts the amplified reception signals into digital signals. The receiving section 230 includes a time gain compensation (TGC) unit (not shown) for compensating attenuation produced while the ultrasound signal passes through the inside of the target object, an analog-to-digital conversion unit (not shown) for converting analog signals into digital signals, and the like.

The processor 120 further includes a data acquiring section 240. The data acquiring section 240 acquires ultrasound data for obtaining a blood flow Doppler image based on the digital signals converted by the receiving section 230. In one embodiment, the data acquiring section 240 performs receive focusing on the digital signals provided from the receiving section 230 to generate receive-focused signals, based on a time delay value for compensating an arrival time of the ultrasound echo signals reflected from a target object of the living body according to the position of the ultrasound transducer 112. In addition, the data acquiring section 240 generates ultrasound data based on the receive-focused signals. In this embodiment, the ultrasound data includes in-phase/quadrature (I/Q) data in the form of a complex number.

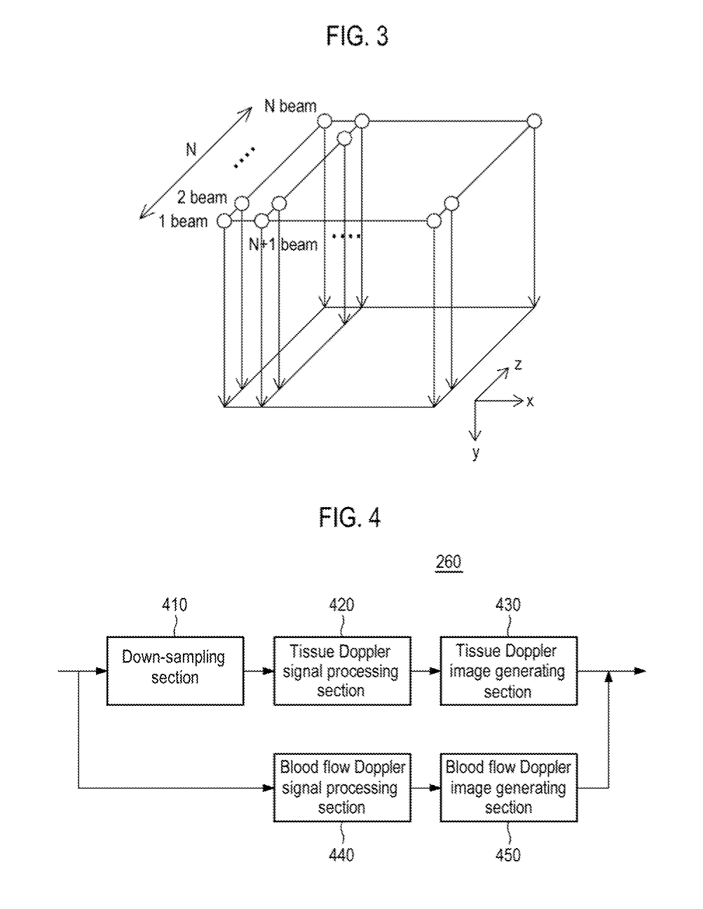

The processor 120 further includes a storage section 250. The storage section 250 stores the ultrasound data provided from the data acquiring section 240. For example, as shown in FIG. 3, the storage section 250 stores ultrasound data (beams) in the z-direction that match the ensemble number and correspond to individual scan-lines forming an ultrasound image. In FIG. 3, N indicates the ensemble number, the x-direction indicates a direction corresponding to the plurality of scan-lines forming the ultrasound image, the y-direction indicates a depth direction, and the z-direction indicates a direction corresponding to the ensemble number.

The processor 120 further includes a Doppler image generating section 260. The Doppler image generating section 260 generates a blood flow Doppler image and a tissue Doppler image corresponding to a region of interest, based on the ultrasound data.

In this embodiment, the Doppler image generating section 260 retrieves ultrasound data from the storage section 250 and generates the blood flow Doppler image indicating movement of blood flow based on the retrieved ultrasound data. Furthermore, the Doppler image generating section 260 performs down-sampling on the ultrasound data retrieved from the storage section 250 and generates the tissue Doppler image indicating movement of the tissue based on the down-sampled ultrasound data.

FIG. 4 is a block diagram schematically showing a configuration of the Doppler image generating section 260 according to an embodiment of the present disclosure. Referring to FIG. 4, the Doppler image generating section 260 includes a down-sampling section 410.

The down-sampling section 410 performs down-sampling on ultrasound data based on a pulse repetition frequency for obtaining the blood flow Doppler image and a pulse repetition frequency for obtaining the tissue Doppler image.

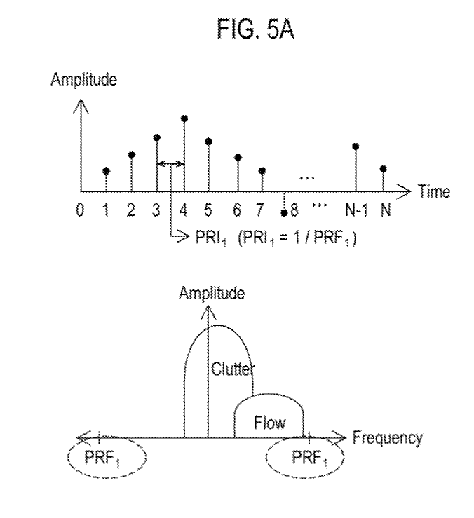

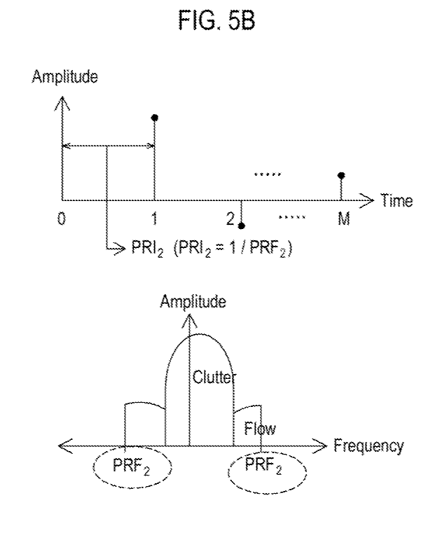

Generally, the velocity of blood flow is three to five times faster than the velocity of tissue. This requires that a pulse repetition frequency for obtaining the blood flow Doppler image is relatively higher than a pulse repetition frequency for obtaining the tissue Doppler image, as shown in FIGS. 5A and 5B. For example, a pulse repetition frequency for obtaining the blood flow Doppler image is 4,000 Hz, while a pulse repetition frequency for obtaining the tissue Doppler image is 1,000 Hz. In FIGS. 5A and 5B, PRF₁ indicates the pulse repetition frequency for obtaining the blood flow Doppler image, PRF₂ indicates the pulse repetition frequency for obtaining the tissue Doppler image, PRI₁ indicates a pulse repetition interval for obtaining the blood flow Doppler image, and PRI₂ indicates the pulse repetition interval for obtaining the tissue Doppler image.

In this embodiment, the down-sampling section 410 calculates a down-sampling rate based on the pulse repetition frequency for obtaining the blood flow Doppler image and the pulse repetition frequency for obtaining the tissue Doppler image. The down-sampling rate can be calculated based on the following equation:

$$DR = \frac{{PRF}_{1}}{{PRF}_{2}} \qquad Equation ( 1 )$$

In Equation (1), DR indicates a down-sampling rate, PRF₁ indicates a pulse repetition frequency for obtaining a blood flow Doppler image, and PRF₂ indicates a pulse repetition frequency for obtaining a tissue Doppler image.

For example, when the pulse repetition frequency PRF₁ for obtaining the blood flow Doppler image is 4,000 Hz and the pulse repetition frequency PRF₂ for obtaining the tissue Doppler image is 1,000 Hz, the down-sampling section 410 calculates the down-sampling rate (DR=4) by applying the pulse repetition frequency PRF₁ for obtaining the blood flow Doppler image and the pulse repetition frequency PRF₂ for obtaining the tissue Doppler image in Equation 1, and performs the down-sampling on the ultrasound data based on the down-sampling rate (DR=4) as shown in FIG. 5B.

The Doppler image generating section 260 further includes a tissue Doppler signal processing section 420. The tissue Doppler signal processing section 420 acquires information indicating movement of the tissue (hereinafter, referred to as “tissue movement information”) by using the down-sampled ultrasound data. The tissue movement information includes information on the velocity and direction of the movement of the tissue.

The Doppler image generating section 260 further includes a tissue Doppler image generating section 430. The tissue Doppler image generating section 430 generates a tissue Doppler image based on the tissue movement information provided from the tissue Doppler signal processing section 420.

The Doppler image generating section 260 further includes a blood flow Doppler signal processing section 440. The blood flow Doppler signal processing section 440 retrieves ultrasound data from the storage section 250 and acquires information indicating movement of the blood flow (hereinafter, referred to as “blood flow movement information”) based on the retrieved ultrasound data. The blood flow movement information includes information on the velocity and direction of the movement of the blood flow.

The Doppler image generating section 260 further includes a blood flow Doppler image generating section 450. The blood flow Doppler image generating section 450 generates a blood flow Doppler image based on the blood flow movement information provided from the blood flow Doppler signal processing section 440.

Referring back to FIG. 2, the processor 120 further includes an image processing section 270. The image processing section 270 performs image processing on the tissue Doppler image and the blood flow Doppler image.

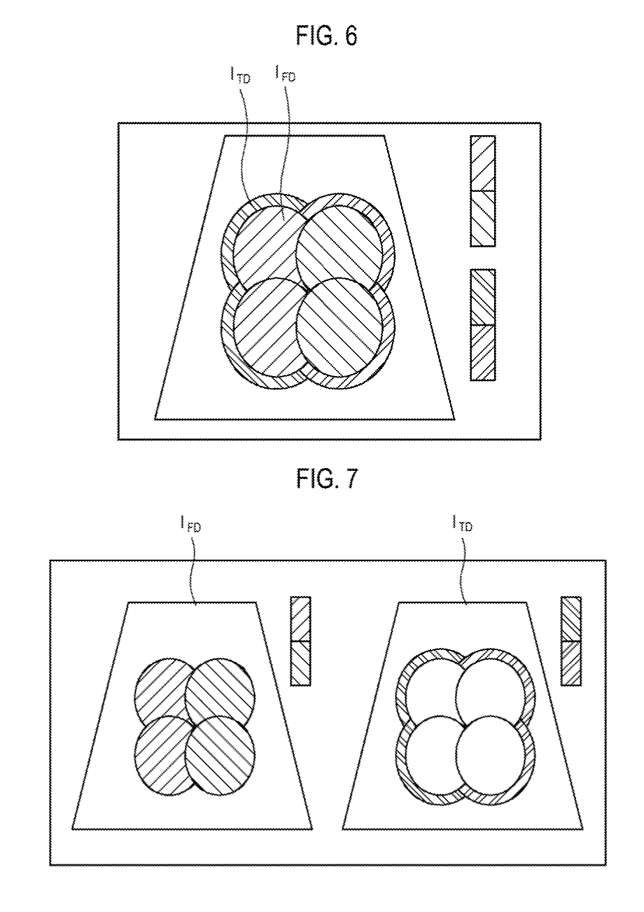

In one embodiment, the image processing section 270 performs image processing to display a tissue Doppler image I_{TD} and a blood flow Doppler image I_{FD} in an overlapped manner as shown in FIG. 6. As an example, the image processing section 270 overlaps the tissue Doppler image I_{TD} and the blood flow Doppler image I_{FD} with each other such that the tissue Doppler image I_{TD} is located over the blood flow Doppler image I_{FD}. As another example, the image processing section 270 overlaps the tissue Doppler image I_{TD} and the blood flow Doppler image I_{FD} with each other such that the blood flow Doppler image I_{FD} is located over the tissue Doppler image I_{TD}. Accordingly, the output section 140 (see FIG. 1) displays the tissue Doppler image I_{TD} and the blood flow Doppler image I_{FD} as shown in FIG. 6 according to a display scheme set by the image processing section 270 (see FIG. 2).

In another embodiment, the image processing section 270 performs image processing to display the tissue Doppler image I_{TD} and the blood flow Doppler image I_{FD} side-by-side so that the tissue Doppler image I_{TD} and the blood flow Doppler image I_{FD} are not overlapping with each other, as shown in FIG. 7. Accordingly, the output section 140 displays the tissue Doppler image I_{TD} and the blood flow Doppler image I_{FD} side-by-side as shown in FIG. 7 according to a display scheme set by the image processing section 270.

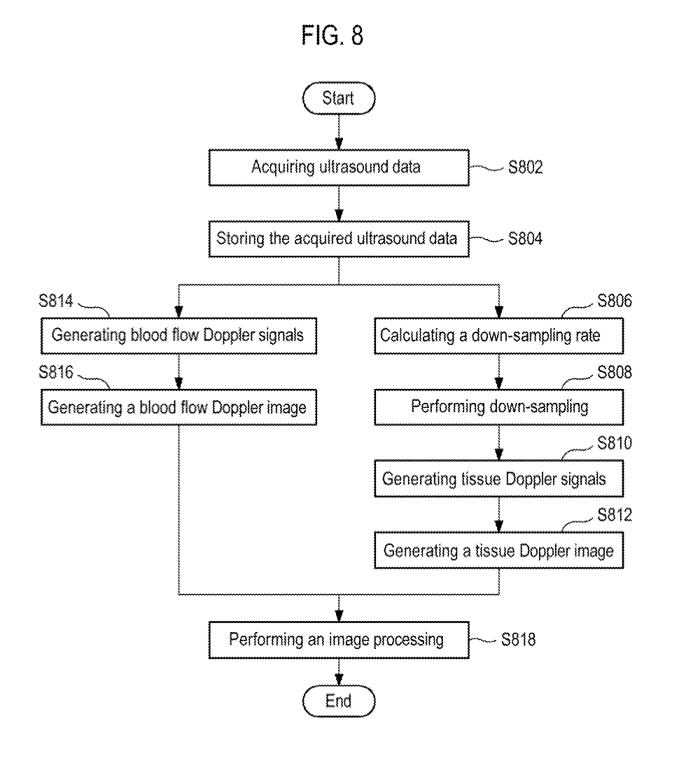

FIG. 8 is a flowchart illustrating a process of simultaneously providing a blood flow Doppler image and a tissue Doppler image according to an embodiment of the present disclosure. Referring to FIG. 8, the processor 120 (see FIG. 1) acquires ultrasound data based on the reception signals provided from the ultrasound probe 110 (see FIG. 1) (S802), and stores the acquired ultrasound data in the storage section 250 (see FIG. 2) (S804).

The processor 120 calculates a down-sampling rate based on a pulse repetition frequency for obtaining the blood flow Doppler image and a pulse repetition frequency for obtaining the tissue Doppler image (S806). The down-sampling rate can be calculated by Equation 1.

The processor 120 retrieves the ultrasound data stored in the storage section 250 and performs down-sampling on the ultrasound data based on the calculated down-sampling rate (S808). The processor 120 generates tissue Doppler signals based on the down-sampled ultrasound data (S810) and generates the tissue Doppler image based on the tissue Doppler signals (S812).

The processor 120 retrieves the ultrasound data stored in the storage section 250, generates blood flow Doppler signals based on the retrieved ultrasound data (S814), and generates the blood flow Doppler image based on the blood flow Doppler signals (S816).

The processor 120 performs image processing on the tissue Doppler image and the blood flow Doppler image (S818). In one embodiment, the processor 120 performs image processing to display the tissue Doppler image I_{TD} and the blood flow Doppler image I_{FD} in the overlapping manner, as shown in FIG. 6. In another embodiment, the processor 120 performs the image processing to display the tissue Doppler image I_{TD} and the blood flow Doppler image I_{FD} side-by-side such that the tissue Doppler image I_{TD} and the blood flow Doppler image I_{FD} are not overlapping with each other, as shown in FIG. 7.

Although it has been described that the process of generating the tissue Doppler image (S806 to S812) and the process of generating the blood flow Doppler image (S814 to S816) are sequentially performed, they are not necessarily limited thereto. In another embodiment, the process of generating the tissue Doppler image and the process of generating the blood flow Doppler image may be performed simultaneously.

Advantageously, the present disclosure can simultaneously provide a blood flow Doppler image and a tissue Doppler image indicating movement of a tissue, particularly, a diastolic function of the heart, in an identical heart cycle.

Further, the present disclosure can provide accurate hemodynamic information on a target object (a patient) suffering from a heart disease of arrhythmia, such as atrial-fibrillation or the like, by simultaneously providing a tissue Doppler image and a blood flow Doppler image.

Furthermore, the present disclosure can simultaneously provide a blood flow Doppler image and a tissue Doppler image by using Doppler signals for obtaining the blood flow Doppler image, thereby eliminating a loss in temporal resolution.

Although the present disclosure has been described and illustrated in connection with the preferred embodiments, it will be readily understood by those skilled in the art that various modifications and changes can be made thereto without departing from the spirit and scope of the present disclosure defined by the appended claims.

What is claimed is:

1. An ultrasound system, comprising:
an ultrasound probe configured to transmit ultrasound signals into a target object having blood flow and tissue, and receive ultrasound echo signals reflected from the target object;
a processor configured to acquire ultrasound data based on the ultrasound echo signals, generate a first Doppler image of the blood flow based on the ultrasound data, perform down-sampling on the ultrasound data to obtain down-sampled ultrasound data, and generate a second Doppler image indicating movement of the tissue based on the down-sampled ultrasound data; and
a display section configured to display the first Doppler image and the second Doppler image.

2. The ultrasound system of claim 1, wherein the ultrasound signals are transmitted according to a first pulse repetition frequency adapted to generate the first Doppler image.

3. The ultrasound system of claim 2, wherein the processor is configured to:
calculate a down-sampling rate based on the first pulse repetition frequency adapted to generate the first Doppler image and a second pulse repetition frequency adapted to generate the second Doppler image; and
perform the down-sampling on the ultrasound data based on the down-sampling rate.

4. The ultrasound system of claim 3, wherein the down-sampling rate is calculated according to the following equation:
DR=PRF₁/PRF₂
wherein DR indicates the down-sampling rate, PRF₁ indicates the first pulse repetition frequency, and PRF₂ indicates the second pulse repetition frequency.

5. The ultrasound system of claim 1, wherein the processor is configured to generate a combined image of the first Doppler image and the second Doppler image and display the combined image on the display section.

6. The ultrasound system of claim 1, wherein the processor is configured to display the first Doppler image and the second Doppler image side by side on the display section.

7. A method of providing a Doppler image of a target object in an ultrasound system, comprising:
transmitting ultrasound signals into a target object having blood flow and tissue;
receiving ultrasound echo signals reflected from the target object;
acquiring ultrasound data based on the ultrasound echo signals;
generating a first Doppler image of the blood flow based on the ultrasound data;
performing down-sampling on the ultrasound data to obtain down-sampled ultrasound data;
generating a second Doppler image indicating movement of the tissue based on the down-sampled ultrasound data; and
displaying the first Doppler image and the second Doppler image.

8. The method of claim 7, wherein the ultrasound signals are transmitted according to a first pulse repetition frequency adapted to generate the blood flow Doppler image.

9. The method of claim 8, wherein performing the down sampling comprises:
calculating a down-sampling rate based on the first pulse repetition frequency adapted to generate the blood flow Doppler image and a second pulse repetition frequency adapted to generate the tissue Doppler image; and
performing the down-sampling upon the ultrasound data based on the down-sampling rate.

10. The method of claim 9, wherein the down-sampling rate is calculated according to the following equation:
DR=PRF₁/PRF₂
wherein DR indicates the down-sampling rate, PRF₁ indicates the first pulse repetition frequency, and PRF₂ indicates the second pulse repetition frequency.

11. The method of claim 7, wherein displaying the first Doppler image and the second Doppler image comprises displaying a combined image of the first Doppler image and the second Doppler image.

12. The method of claim 7, wherein displaying the first Doppler image and the second Doppler image comprises displaying the first Doppler image and the second Doppler image side by side.

13. A non-transitory computer-readable storage medium comprising instructions that, when executed by a processor, cause the processor to perform operations of:
acquiring ultrasound data based on ultrasound echo signals reflected from a target object having blood flow and tissue;
generating a first Doppler image of the blood flow based on the ultrasound data;
performing down-sampling on the ultrasound data to obtain down-sampled ultrasound data;
generating a second Doppler image indicating movement of the tissue based on the down-sampled ultrasound data; and
displaying the first Doppler image and the second Doppler image.

14. The computer-readable storage medium of claim 13, wherein the first Doppler image is generated based on a first pulse repetition frequency.

15. The computer-readable storage medium of claim 14, wherein performing the down-sampling comprises:
calculating a down-sampling rate based on the first pulse repetition frequency adapted to generate the first Doppler image and a second pulse repetition frequency adapted to generate the second Doppler image; and
performing the down-sampling upon the ultrasound data based on the down sampling rate.

16. The computer-readable storage medium of claim 15, wherein the down-sampling rate is calculated according to the following equation:
DR=PRF₁/PRF₂
wherein DR indicates the down-sampling rate, PRF₁ indicates the first pulse repetition frequency, and PRF₂ indicates the second pulse repetition frequency.

17. The computer-readable storage medium of claim 13, wherein displaying the first Doppler image and the second Doppler image comprises displaying a combined image of the first Doppler image and the second Doppler image.

18. The computer-readable storage medium of claim 13, wherein displaying the first Doppler image and the second Doppler image comprises displaying the first Doppler image and the second Doppler image side by side.
